(19) United States
(12) Patent Application Publication
(10) Pub. No.: US 2020/0390357 A1
(43) Pub. Date: Dec. 17, 2020

(54) EVENT RELATED BRAIN IMAGING

(71) Applicant: Neurofeedback-Partner GmbH, Munchen (DE)

(72) Inventor: Thomas FEINER, Munchen (DE)

(21) Appl. No.: 16/440,501

(22) Filed: Jun. 13, 2019

(51) Int. Cl. A61B 5/0478; A61B 5/00; A61B 5/0484

(52) U.S. Cl. CPC A61B 5/0478; A61B 5/7425; A61B 5/048; A61B 5/0484; A61B 5/0042; A61B 5/6803

(57) ABSTRACT

A system and method to generate, store, retrieve, adjust, arrange, link, and display a series of medical images. Further, a system and method to do the same for a plurality of series of images that are different but related, and to select, link, coordinate, combine, and simultaneously display more than one of the series of images.

### BACKGROUND

Broadly speaking, there are two types of brain imaging-based tests, called normative and ipsative. Normative tests identify differences in images of the responses of multiple subjects' brain's to similar stimuli. They can be used for example to compare one subjects' response to a stimulus to the average response of many subjects to the same stimulus, in one or more testing events for each subject. So for example, a normative personality test compares measurable personality characteristics of individuals against patterns of normality based on multiple people's test results. Such personality testing may allow testers to compare a particular subject's results with other subjects' results, possibly grouped by populations having known characteristics.

In contrast, ipsative tests are used to identify changes in a series of images of a single subject's brain in response to a stimulus in one or multiple testing events over time. This can be used, for example, to determine a subject's brain's response to similar events over time, such as the brain's response to changes in sleep patterns, changes in work pace or procedures, reactions to certain people, and the like. The ipsative approach detects changes in a test subject's own responses. It does not compare a test subject's responses to other people's test responses.

Thus, ipsative assessment is based on a test subject's own previous performance or state, and not based on performance or state of others, or against any other external criteria and standards. Normative and ipsative techniques can also be combined. For example, in an educational context, a normative measure may be used to establish a baseline of acceptable performance, for example, in a test of skill or knowledge. A subject may thereafter be assessed individually, and progress can be shown by assessing improvements in their own performance or state, and not against a norm, in cases where a threshold standard must be met to achieve a successful outcome, ipsative feedback can also advantageously be combined with normative feedback. Benefits to a student of using an ipsative approach can include avoiding or supplementing standards- and criteria-referenced assessment, which can be demotivating for a student whose performance is below the average, in contrast, ipsative assessments emphasize the progress a student is making, which can be more motivating. For example, ipsative feedback can help by highlighting areas where a student is not making sufficient progress to meet the standard, and can help focus their efforts to improve those areas. Such feedback can also help high performing students to achieve even more. Moreover, ipsative assessment allows students to self assess and become more independent.

Benefits to educators can include providing feedback regarding where students' efforts are most likely to be effective. Students may also be more likely to act on ipsative feedback that emphasizes progress, rather than conventional normative feedback that emphasizes deficiencies. Further, focusing on progress identifies students who are progressing, albeit more slowly than the norm, and also identifies those who are not. This permits learning resources to be more effectively allocated.

Ipsative techniques can also be used in a medical context. However, in a medical context a disadvantage of the ipsative approach can be that an assessor needs to have access to a subject's past assessments to track progress. These may not be available. Furthermore, different characteristics of a subject may need to be linked in an assessment scheme. This may be difficult if the characteristics have very different progressions, status, or outcomes. For example in a medical imaging context, images of various kinds for a single patient are typically generated separately, and are viewed one at a time. Thus, to view one or more series of images requires them to be retrieved from storage, adjusted individually, and arranged by hand to show the characteristics of interest. This process is even more complicated if the images were made to different scales, or if different colors were used to indicate the same characteristic in different images. This way of viewing series of images is time consuming and prone to error. It is desirable to be able to generate, store, retrieve, adjust, arrange, and view a plurality of medical images quickly and easily.

### SUMMARY

A system and method to generate, store, retrieve, adjust, arrange, link, and display a series of medical images. Further, a system and method to do the same for a plurality of series of images that are different but related, and to select, link, coordinate, combine, and simultaneously display more than one of the series of images.

It is to be understood that both the foregoing general description and the following detailed description are exemplary and explanatory and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate disclosed embodiments and/or aspects and, together with the description, serve to explain the principles of the invention, the scope of which is determined by the claims.

In the drawings:

FIG. 1 is a block diagram of an exemplary brain scanning system comprising an exemplary embodiment, in accordance with the disclosure.

FIGS. 2A and 28 are exemplary screen elements presented on a display, in accordance with the disclosure.

FIG. 3 is a session analyzer screen in accordance with the disclosure.

FIG. 4 is a screen on which a user can change individual parameters as desired for a session, in accordance with the disclosure.

FIG. 5 is a screen showing post processing performed by the system which includes sLoretta analysis, in accordance with the disclosure.

FIG. 6 shows an image being cropped in accordance with previously defined parameters for use in an automatically generated report, in accordance with the disclosure.

FIGS. 7A and 7B illustrate a synchronization feature of an embodiment, in accordance with the disclosure.

FIG. 8 is a flow diagram illustrating an exemplary method embodiment, in accordance with the disclosure.

FIG. 9 is a block diagram of a computing system in which aspects of exemplary embodiments may be realized, in accordance with the disclosure.

### DETAILED DESCRIPTION

It is to be understood that the figures and descriptions provided herein may have been simplified to illustrate aspects that are relevant for a clear understanding of the herein described processes, machines, manufactures, and/or compositions of matter, while eliminating, for the purpose of clarity, other aspects that may be found in typical devices, systems, and methods. Those of ordinary skill in the pertinent art may recognize that other elements and/or steps may be desirable and/or necessary to implement the devices, systems, and methods described herein. Because such elements and steps are well known in the art, and because they do not facilitate a better understanding of the present disclosure, a discussion of such elements and steps may not be provided herein. However, the present disclosure is deemed to inherently include all such elements. variations, and modifications to the described aspects that would be known to those of ordinary skill in the pertinent art.

It will be readily understood that the components of the present invention, as generally described and illustrated in the figures herein, may be arranged and designed In a wide variety of different configurations. Thus, the following detailed description of the method, apparatus, and system embodiments as represented in the attached figures is not intended to limit the scope of the invention as claimed, but is merely representative of exemplary embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

A variety of embodiments will now be described. These embodiments are provided as teaching examples and should not be interpreted to limit the scope of the invention. Although specific details of the embodiments are presented, these embodiments may be modified by changing, supplementing, or eliminating many of these details.

It is well known that so-called functional neuroimaging may be used to investigate brain activity involved during certain cognitive and other brain functions. This can result in a better understanding of where in the brain the corresponding processes take place, and their role in brain function. Moreover, various imaging technologies, combinations of technologies, and imaging techniques may contribute to a better understanding of the brain, including normal and abnormal brain function.

Some technologies, such as electroencephalogram (EEG) technology, provide fast response times and can track brain activity in real time. However, such technologies may not provide good spatial resolution. Conversely, other technologies, for example Computed Tomography (CT) scanning, Magnetic Resonance Imaging (MRI), and others, allow brain structure and function to be examined with good spatial resolution, but may not be able to keep up in real time with the brain's processing. Accordingly in embodiments, different brain imaging technologies and approaches may be combined to obtain improved imaging of changes in the brain that occur responsive to various stimuli.

In practice, physical or mental stimuli may be provided to a subject while the imaging of the subject's brain is taking place. For example, an array of EEG sensors placed on the subject's scalp may track the activity of multiple distributed neuronal processes as the subject responds to a visual or other sensory cue by hitting a button. At the same time, techniques such as low-resolution brain electromagnetic tomography (LORETA) may be used to image certain brain activity, such as to detect areas in the brain that fire in connection with a visual cue and related motor functions. Images and data collected from a subject, in real time may be correlated and used to reveal aspects of brain activity that may not be apparent otherwise. The images and data may be correlated, combined, and processed using any number of approaches. The embodiments and aspects described herein organize, manage, and simplify these data, images, and processes.

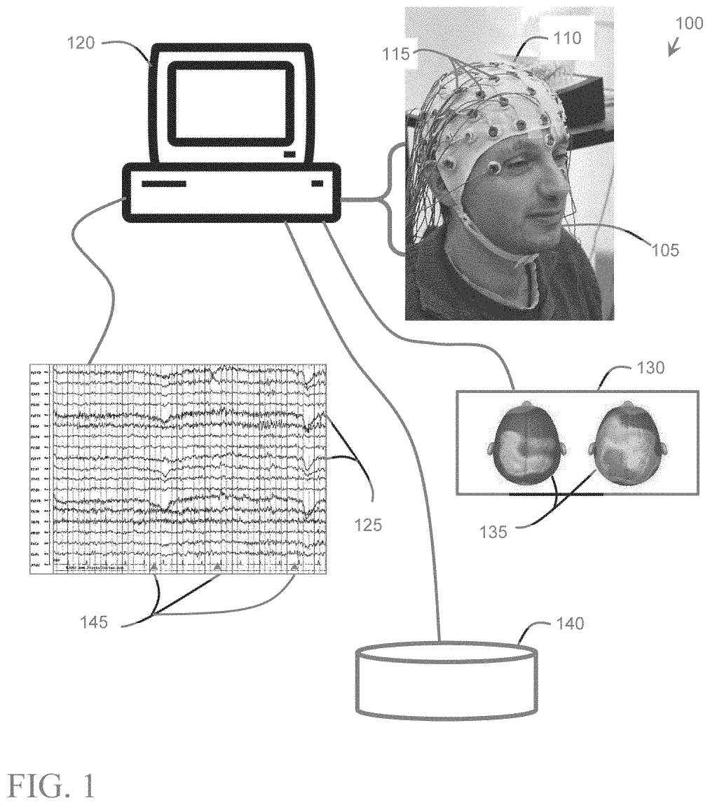

FIG. 1 illustrates aspects of an exemplary embodiment of a system 100. As shown, a test subject 105 is prepared to have his brain imaged, or an EEG graph made, or both. The subject is shown wearing a headpiece 110 comprising a plurality of electroencephalogram (EEG) sensors 115, although other sensor arrangements may additionally or alternatively be used. These detect brain wave patterns at the location of the sensors. Commonly, the sensors have small metal discs with thin wires (electrodes) placed on or near the skin. These detect electrical signals indicative of localized brain activity, and convey the signals to a computer 120 or other electronic device to record the activity. The sensed brain activity can be graphed 125, and may form recognizable patterns. Both normal and abnormal patterns may be identifiable. For example, EEG patterns may indicate the subject has a seizure disorder. EEGs may also be used to identify causes in the brain of certain conditions, such as sleep disorders and changes in behavior.

Neuroimaging modalities other than EEG also exist to study brain function. These include, for example, magnetoencephalography (MEG), functional magnetic resonance imaging (fMRI), positron emission tomography (PET), nuclear magnetic resonance spectroscopy (NMR or MRS), electrocorticography (ECoG), single photon emission computed tomography (SPECT), near-infra red spectroscopy (NIRS), event-related optical signal (EROS), and the like. In some cases, the subject may be placed in or near an imaging apparatus 130, which may generate an imaging field or radiation. Thereby, the brain's responses to stimuli may be captured and displayed, 135. Such modalities may provide different sensitivity and/or better spatial sensitivity than an EEG when represented graphically.

In embodiments, data and images can be stored in storage device 140, which may be coupled directly to the data- or image-producing equipment either locally or remotely (such as through a network), or indirectly via computerized equipment coupled to one or more the equipment.

In an exemplary operation, the test subject 105 may be presented with or subjected to one or more stimuli. The stimuli may be caused or controlled by the system 100. Alternatively or in addition, the stimuli may be initiated by a stimulus driver or the like external to the system, that may be coupled to system 100 to provide the system with information of the stimuli. In either case, the system may mark the EEG graph with the time of the stimuli, 145, and store the times of the stimuli in association with information of the EEG in storage device 140. The stored data may be sufficient to allow reproduction of the EEG graph with stimuli time marks with reasonable fidelity. For example, one or more of the channels (i.e., the lines 125 drawn on the EEG chart) of the EEG may be sampled at a select or default frequency, e.g., every 10 ms, or every 100 ms, or another frequency. Similarly, images 135 may be sampled at the same or a different select or default frequency and stored. Alternatively or in addition, data from which the images may be reproduced with reasonable fidelity may be stored. In embodiments, the stored images and/or image data may be correlated with the stored EEG graphs or data, stimulus timing data, or the like.

In embodiments, system 100 can obtain, store, and reproduce Event Related Imaging (ERI). With ERI it is possible to see different activation patterns in the brain in response to various stimuli. Brain images, or brain sensing data that is amenable to being presented as images, can be obtained from one or more imaging modes, devices, or sensors as previously described. These can include, for example, one or more of infrared (IR) sensors, electroencephalogram (EEG) sensors, low resolution electromagnetic tomography (LORETA), standardized LORETA (sLORETA), functional Magnetic Resonance Tomography and others. The images or data can be marked or correlated with stimulus timing data and/or other stimulus data, stored, and retrieved. In embodiments, system 100 sends and stores the stimulus information with the recorded brain data and/or images in real time.

In embodiments, the images can be retrieved, modified, arranged, and presented at any time during or after the stimulus session in a process referred to herein as post processing. In post processing a plurality of images are obtained as a series associated with a corresponding stimulus. There may be a select or predetermined number of images or image data, gathered at select or predetermined intervals. For example, from each stimulus there may be 6-12 images taken per second. In an embodiment, the images may be taken as screenshots of image or other graphical information presented on a computer monitor. In embodiments, image data can be obtained and marked sequentially in real time by a computerized platform running software operative to do so. The software may display the images on a monitor in timestamp order showing brain activation locations. The brain activation imagery may be stored with the timestamp information and correlated to the corresponding stimuli.

In an embodiment, to do the correlation the software may obtain the pictures from a folder on a local or network drive, where they may have been stored by EEG-recording software, picture-generating software, or the like. The software may be comprised in system 100. The correlation software may collect the images and modify them for display purposes. For example, the correlation software may put the correct corresponding time-stamps on them. It may crop all of the images to show only a selected or pre defined portion of interest. In an embodiment, the software may put an original or modified set of images into a document or spreadsheet template, for example, in a list where the corresponding stimuli are listed. For example, an Excel spreadsheet may be generated. The user then has the stimulus and the brain pattern images corresponding to the stimulus, organized and saved automatically by the software.

In embodiments, the software can be used to repeat the process, one or more times, using the same original images and/or data. For example, the system may include a graphics tablet and stylus or the like that can be used to select another region of interest. For example, images such as a series of sLoreta images may be identified, and an area to crop each image of the series can be defined, or selected from a plurality of pre-defined areas. In this way, post processing can be performed in multiple assessments using data and imaging of the same subject. These may be, for example, images and data from a single imaging window of time, with the subject physically present in only that one window. That is, data and imaging gathered from the subject in a single visit can be stored and used in multiple assessments, including in combination with data and imaging produced and stored at an earlier or later time. Post processing event-related imaging assessments can thus be done quickly and easily using disclosed embodiments.

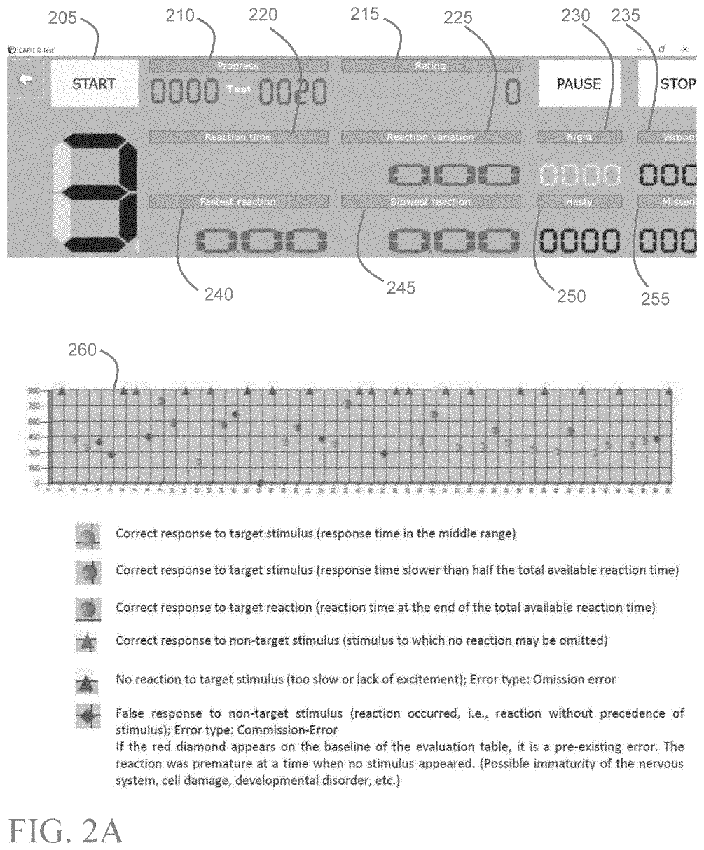

In a currently preferred embodiment, system 100 can comprise a computing system running software. The computer and software can be coupled directly or indirectly to brain data generating and/or brain image generating devices. An exemplary embodiment can include, for example, a test screen for initiating and managing an assessment session, for example as shown at the top of FIG. 2A. The screen shown includes various graphical elements that can be controlled by a user, for example by clicking a mouse to select a button. In the exemplary embodiment illustrated, those elements can include:
a start button 205 to start the test;
a pause button to pause the test;
a stop button 205 to stop the test;
a progress section 210 that provides information about the test progress;
a rating section 215 that displays a test rating;
a reaction time field 220 that displays the subject's reaction time;
a reaction variation field 225 that displays variation in the subject's reaction time;
a right field 230 that displays the number of right actions;
a wrong field 235 that displays the number of wrong actions;
a fastest reaction field 240 that displays the fastest reaction time;
a slowest reaction field 245 that displays the slowest reaction time;
a hasty field 250 that displays the number of hasty reactions; and
a missed field 255 that displays the number of missed reactions.

Exemplary evaluation criteria can include the following, although other criteria may alternatively or additionally be used.
standard deviation—based on the difference between fastest and slowest response (value should be as low as possible, the lower the more capable the client)
reaction variation—based on fluctuation (e.g., variance) of the reaction times (lower values indicate stable performance)
Reaction mean (average)—Average reaction time
OK—Number of correct responses to a target stimulus
NOK (not OK)—Number of incorrect responses to a target stimulus (Comission Errors)
Hasty—Number of anticipatory responses to a non-existent stimulus
Missed—Number of missed reactions (Omission-Errors)
Rating—Total score (over multiple sessions)

In embodiments, to facilitate administering a test session a separate window may open (not shown) in which images may be shown for testing purposes. The subject reacts, or does not react, to these Images in so-called trials. In embodiments, other modifications (for example Dual Screen) can be made, for example in a settings menu (not shown).

In an exemplary testing scenario, to begin a test, a countdown may appear in a test window, and a brief explanation of the test may be displayed. A test session is a sequence of individual trials, and an assessment window of time (i.e., in which a test subject participates in the test) may include a plurality of test sessions. In embodiments, the system saves session events as a contiguous unit, which can be viewed in the evaluation and statistics window. A subject's reaction may be carried out by pressing a key on a keyboard, or by means of a physical push-button coupled to the system, for example. When the subject reacts to an image, the system records information of the reaction in the fields explained above, and a graphical representation of the subjects reactions is shown in a graphics section 260, as shown in the middle of FIG. 2A. The graphics section uses different symbols for different types of trials or responses, explained in the key at the bottom of FIG. 2A.

From the responses information, an evaluation score may be calculated for comparing the results obtained in one or more test sessions, as illustrated in FIG. 2B. In the figure, a maximum positive or negative point value is set for each type of response, As shown, each type of response has a maximum of 10,000 points, either positive or negative. The point value of each actual response may then be determined, for example as a fraction of the maximum. In an embodiment, evaluation subscores may be calculated by adding together the values for each type of response, which may then be added together and compared with corresponding subscore sums of other test sessions, for example. A test session evaluation score may be calculated by adding together the subscores of the test session, which may then be compared with the test session evaluation scores of other test sessions. Other operations may additionally or alternatively be used, for example statistical analyses such as variances and confidence levels. Only sessions having the same elements, maximum set point values, analyses, and the like, should be compared directly with each other.

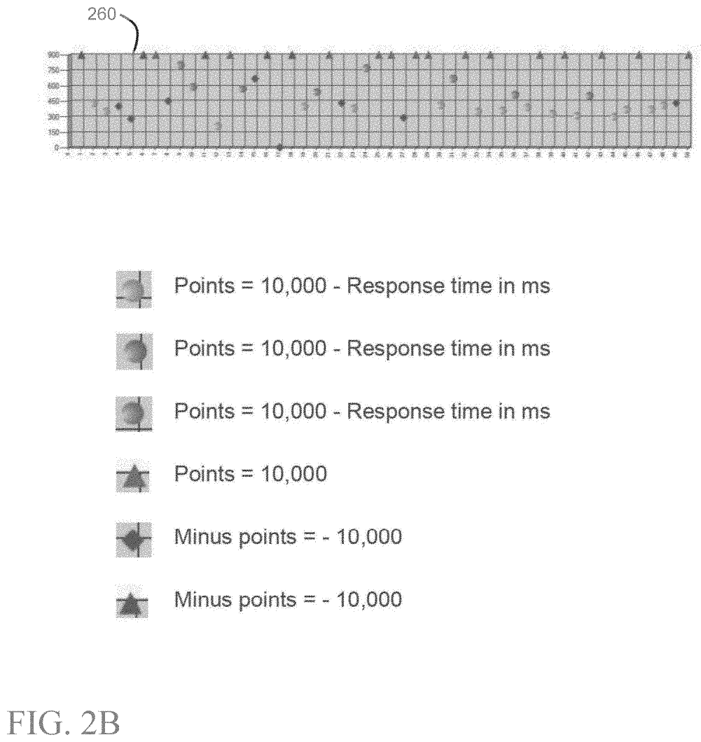

In embodiments, a session analyzer screen can be automatically or selectively invoked, as shown in FIG. 3. Here a system user can view the results of the respective tests for one or more selected subjects, and save them in a preferred or selected format. For example, a user may save test results for a single subject as comma separated values (CSV) in a flat file, or as columns or rows of data in an Excel file, which may be generated from a template. In embodiments, a plurality of test results may be graphed together in a single graphic and viewed simultaneously, for example as a 3D plot comprising a series of test sessions, each session drawn as a line in three dimensions.

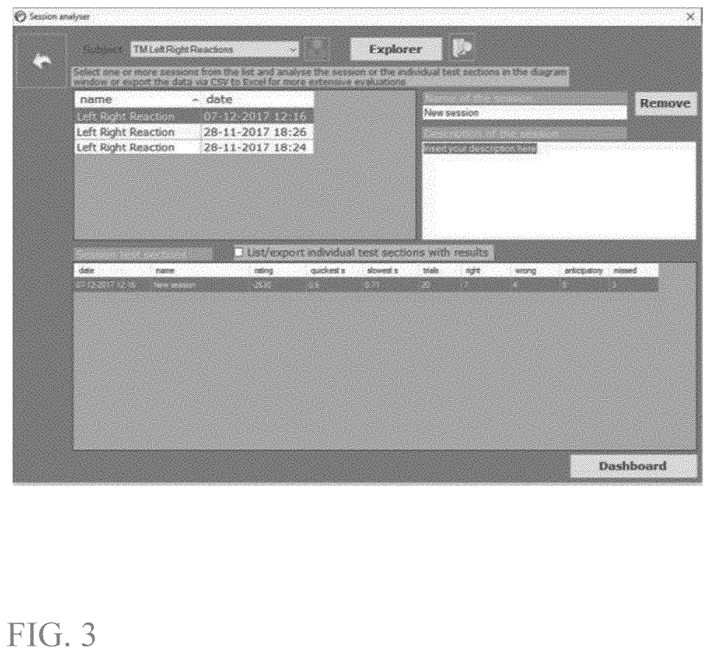

A user can modify the system to produce a variety of sessions, in embodiments, the user can change individual parameters (such as response times and number of trials) as desired, using one or more screens provided for this purpose, such as the one shown in FIG. 4. By filling in the fields shown, a user can change the name and description of a selected test, as well as the number of trials, countdown to test start, maximum allowed response time, interstimulus interval, and number of false positives, although other fields may alternatively or additionally be used. The user can also adjust and change images, numbers, and words that appear in the test.

In embodiments, a template of an Excel output file as described previously may include formulas referring to specific cells for calculations. In embodiments, the stimulus events can include presenting questions to be answered by the test, subject. Questions, answers, and other text may also be contained in the Excel file in a predetermined format. For example, the format can be:
<left question>; <right question>; <type of inquiry>; <range>. Illustratively, relevant test questions, types, and ranges can include:
   Can think clearly; difficulties with thinking; 1; 7
   Solve problems creatively; difficulties with problems; 1; 7
   Can plan well; difficulties in planning, 1; 7
   Can decide well; cannot make decisions; 1; 7
   Can organize well; cannot organize well; 1; 7
   Attention to details; difficulties with details 1; 7

In embodiments, the actual evaluation may be carried out within Excel on the basis of the formulas stored there in a copy of a default or a select template. The results of a test can be written in the correct template. The template may be set up with formulas and text, as described above, and may automatically perform calculations and the like needed to complete an evaluation. In addition, the template may include predefined graphs making reference to ranges of imported data points, predefined print areas and format information, and the like, to facilitate producing a test report.

Post-processing includes obtaining image files, such as images generated during a test. In an embodiment, the imaging apparatus can be set to produce a default or selectable number of images for the brain aspect being evaluated. For example images, or data from which images may be produced, can be generated and captured at a default rate of 8 images per second. The apparatus may also be set to capture images at a different default or selected rate if appropriate. Alternatively or additionally, previously recorded data and images can also be used by the system.

The correlation of test stimuli to the images may also be prepared using a template, such as an Excel template. A copy of the template can be generated and populated with files stored in a default or selected directory containing information and images of the test subject.

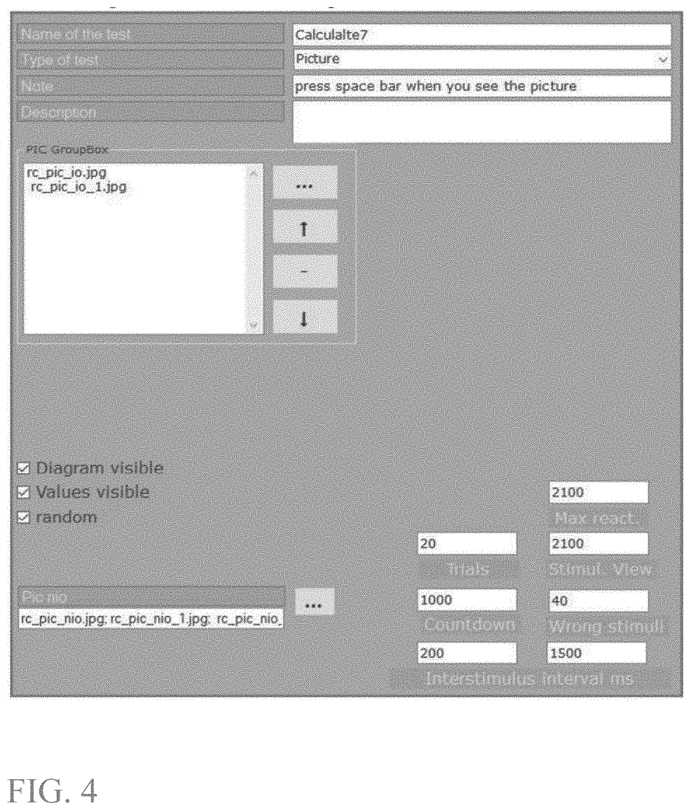

In embodiments, post processing performed by the system can include sLoretta analysis. FIG. 5 shows an exemplary screen shot that may be used for this purpose. Tests must be based on the data types “Image” and “Text”, and as session type “Single”. A control file may be created and stored in a directory set up for the test subject. An exemplary format may be:
<Timestamp>_XPERT-PP.PPX
   For example:
2017.02.19_20.03.59_XPERT-PP
   The user may be prompted to make a selection of the relevant image information (e.g., one or more series of jpg files). The selected files may be placed in a default or selected directory for use in the sLoreta analysis, and may be used in sLoreta analysis well known in the art. When processing is complete, report may be generated saved in the subject's directory. This can be viewed immediately or at a later time. The report may be generated, for example in Excel, in accordance with a predefined template or a select one of a plurality of such templates.

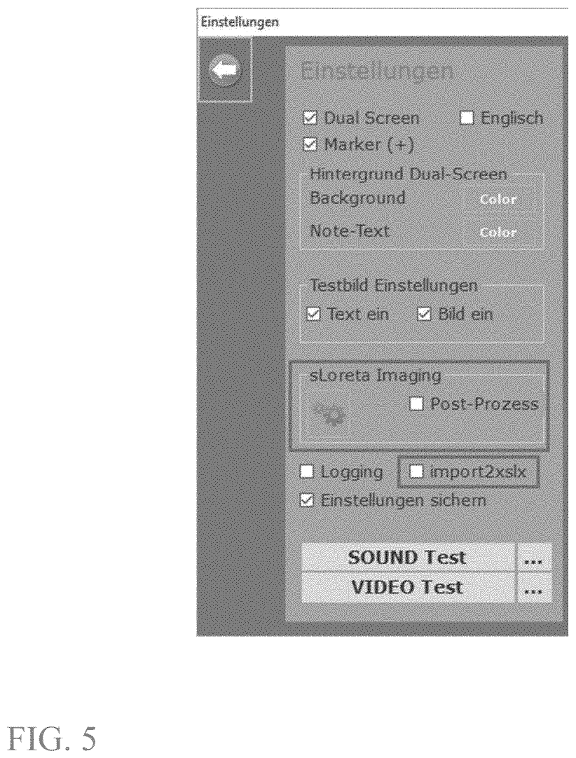

In embodiments, the images displayed and used in the evaluation may be automatically cropped for use in the report by calling any appropriate imaging software module or routine, and defining an area to crop, as illustrated for example in FIG. 6.

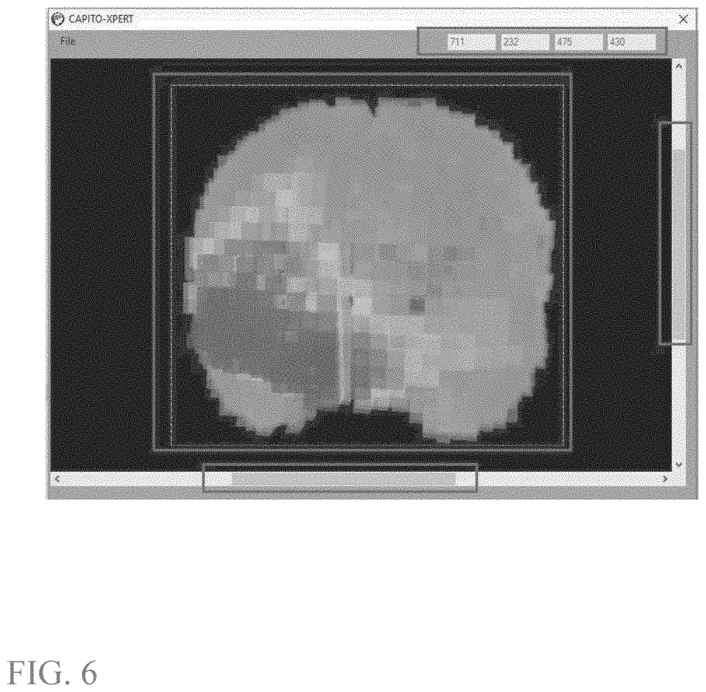

FIGS. 7A and 7B are screens that may be presented on a display in exemplary embodiments. In FIG. 7A, three lines are shown plotted on a graph. The top line, labeled, EEG Oz-A1, is a stimulus signal, which may be controlled by or detected in an embodiment. The stimulus may be any type of signal that may be sensed by a subject of a study, such as an image, video or other visual stimulus, a verbal or other auditory stimulus, or a tactile signal for example. The center line, labeled EEG AUX1A-AUX1R, is a response signal from the subject, such as may be received from a sensor attached to the subject.

One of the characteristics of such studies is that the response signal is received after a delay. The delay may be due to latency in the system, or may be attributable to the subject's biological response time, for example, in embodiments, the presentation of the stimulus signal can be advanced or delayed, such as by advancing or delaying the moment when the stimulus will be transferred to the com-port, which may be coupled to an EEG recording unit, for example. In embodiments, the stimulus signal can thereby be delayed to match the delay in the response, so that the start of the stimulus signal is synchronized with the start of the response signal. This can make the signals presented on the display easier to interpret because they are coordinated. In FIG. 7A, the left side of the figure shows the stimulus and response signals wherein the abscissa (x axis) is time, before the signal display modification. As shown, the delay is identified as the space between two arrows labeled “Screen, Display” (i.e., the stimulus signal) and “Arrival of the signal in the hardware (recording device)” (i.e., the response signal, which may be received directly from a sensor, or from a signal recording device such as an EEG). The right side of the figure shows the graph with a delay added to the stimulus signal, so that stimulus signal coincides with the response to the stimulus. This can make the graph of the two signals easier to interpret.

FIG. 7B is another screenshot showing stimulus signal (top) and response signal (bottom) after the stimulus display is adjusted to coincide in time with the response signal. As shown, the timing of a change in the stimulus signal (here, changing the display between black and white) coincides with the response (an abrupt dip and rebound). Subtle variations in the response to the stimulus may be easier to recognize than if the signals were not coordinated. In particular, a very stable and accurate signal timing presentation is provided. This may help researchers to save time and effort, because precise stimulus-response presentation is extremely important for neuroscientific research.

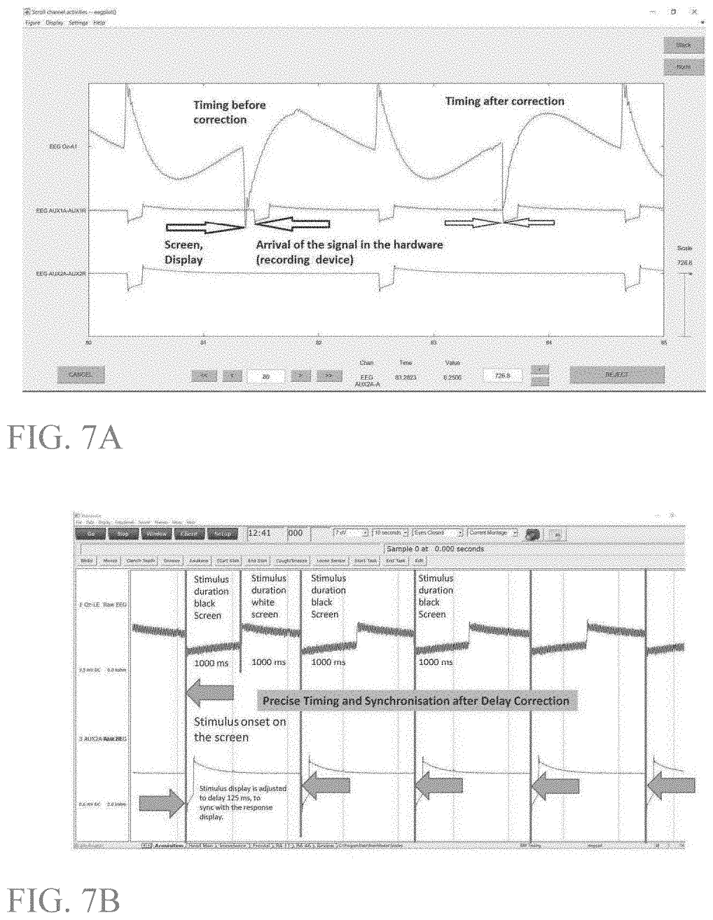

FIG. 8 is a flow diagram illustrating an exemplary method embodiment. At the beginning of a test session, the system begins recording a graph of a subject's brain activity, such as an EEG, 702. Stimulus events are provided to a subject, 704. With each stimulus event, the time the stimulus occurred is marked on the brain activity graph, 706. Brain activity data and other stimulus information, such as time-related information, is gathered and stored in real time, 708. In addition, an imaging device such as an MRI or fMRI, may be set to generate brain images at a select frequency for a select period of time after the stimulus is provided, showing brain activation caused by the stimulus, 710. The EEG graph produced and images generated can be correlated with the stimulus timing, 712, and time stamped 714. The images may be arranged and displayed in their entirety, or the images can be cropped to show a particular area of interest, 716. The images may also be processed by resizing, recoloring, sharpening or softening, or the like, to highlight particular features of interest, 718. The images can be arranged and presented, for example in a spreadsheet along with related information such as the corresponding stimulus that was applied when the images were taken, 720. In a currently preferred embodiment, system software can create a path to a folder on the computer where images from an assessment or experiment may be stored. These images may be selected by the software and automatically transferred into an Excel spreadsheet. The Excel spreadsheet may be automatically created by the software, and arranged so the Excel spreadsheet shows the stimuli and the response images in the exact same order the subject has seen them or responded to them. A report can then be generated, preferably in accordance with a predefined template, containing relevant information obtained from the testing.

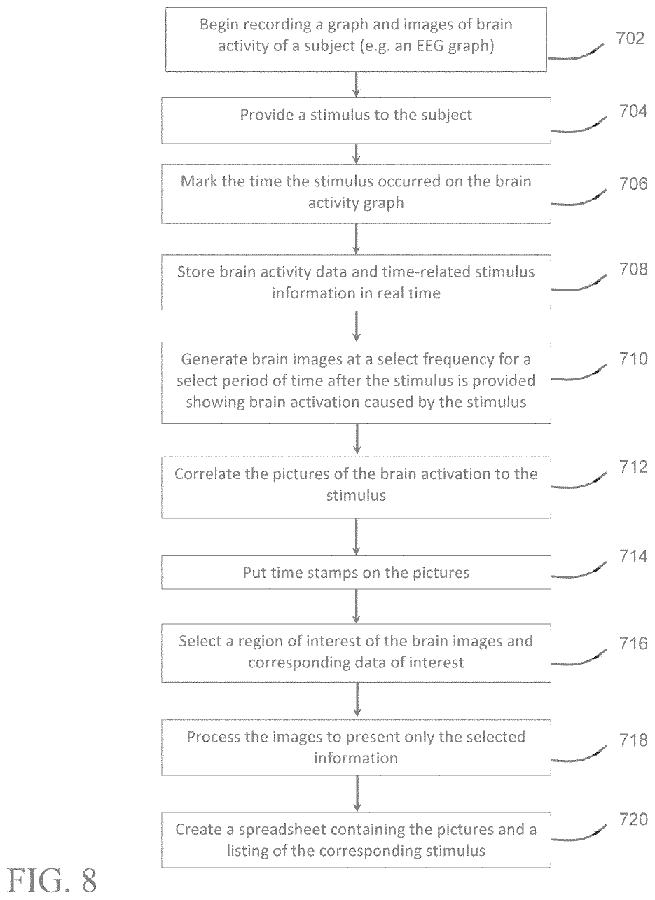

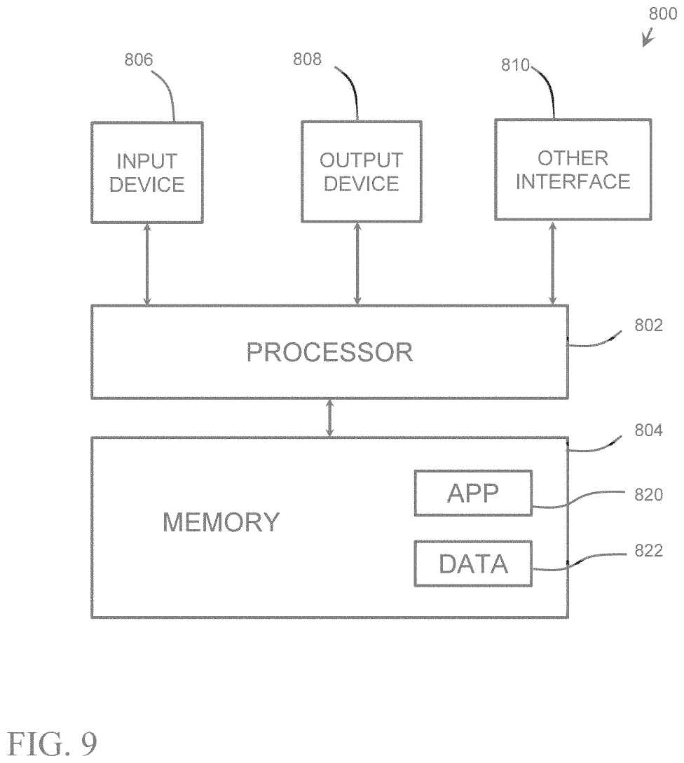

FIG. 9 illustrates a system 800 that implements some or all of the techniques described herein. As shown, the system 800 may include a processor 802 coupled to a memory 804, an input device 806, an output device 808 and an interface 810. Although a single input device is illustrated, a plurality of input devices can be included, such as a keyboard, mouse, graphics tablet, EEG or brain image generating devices, and the like. Similarly, although a single output device is illustrate, a plurality of output devices can be included, such as a flat panel display, a printer, a hard drive or other storage device, and the like. The memory 804 may comprise volatile memory and/or non-volatile memory, and contains an ipsative assessment application 820 that is executed by processor 802. Application 820 causes the system to perform some or all of the actions described above. For example, application 820 may include, or otherwise call, an image modification module, for example using an application programming interface (API) of the module. This may be done when resizing images as described above. The interface 810 may provide the system 800 with connectivity to a network. Historical image data sets processed by the ipsative assessment application 820 may be stored in memory 804. Images and data may be stored in memory 822, in a coupled hard drive or the like, or on a network drive to which access is provided to the system 800 via the network interface 810.

The above discussion is meant to be illustrative of the principles and various embodiments of the present invention. Numerous variations and modifications will become apparent to those skilled in the art once the above disclosure is fully appreciated. The following claims embrace all such variations and modifications.

Although the invention has been described and illustrated in exemplary forms and use cases with a certain degree of particularity, it is noted that the description and illustrations have been made by way of example only. Other usage cases are possible, such as marketing, neuromarketing, court trials, architecture, psychotherapy, counseling, and the like. Moreover, numerous changes in the details of construction, combination, and arrangement of parts and steps may be made without deviating from the scope of the invention. Accordingly, such changes are understood to be inherent in the disclosure. The invention is not limited except by the appended claims and the elements explicitly recited therein. The scope of the claims is to be construed as broadly as the prior art will permit. It should also be noted that all elements of all of the claims may be combined with each other in any possible combination, even if the combinations have not been expressly disclosed.

What is claimed is:

1. A computer-implemented method of Event Related Imaging (ERI), comprising automatedly:
recording time-related information of a stimulus event provided to a subject;
obtaining a series of images of the subject's brain's reaction to the stimulus event;
correlating the series of images with the time-related information of the stimulus event; and
arranging and displaying the series of images in time sequence order.

2. The method of claim 1, further comprising:
obtaining a plurality of series of images of the subject's brain's reaction to a corresponding plurality of stimulus events of a test session;
generating an EEG graph during the test session; and
marking the time of each of the plurality of stimulus events on the EEG graph.

3. The method of claim 1 further comprising automatedly:
generating the series of images by, at regular intervals beginning no later than the start of the stimulus event and continuing for a predetermined length of time:
generating an image and a corresponding time stamp; and
storing the Image in conjunction with the time stamp; and
arranging the images by embedding each of the images in a spreadsheet in time sequence order in conjunction with the corresponding time stamp.

4. The method of claim 3 further comprising:
generating each of the images from image data obtained at a corresponding one of the regular intervals; and
storing at least one of the image and the corresponding image data.

5. The method of claim 4, wherein the data are obtained from one or more electroencephalogram (EEG) sensors, infrared (IR) sensors, low resolution electromagnetic tomography (LORETA), standardized LORETA (sLORETA), computed tomography (CT) scanning, and functional Magnetic Resonance imaging (fMRI).

6. The method of claim 4, further comprising:
receiving a selection of a region of interest of a brain image; and
for ones of the plurality of the brain images:
generating a copy of the brain image; and
cropping the copy in accordance with the region of interest; and
arranging the cropped images by embedding each of the cropped images in a spreadsheet in time sequence order in conjunction with the corresponding time stamp.

7. The method of claim 1, further comprising:
obtaining a second series of images of the subject's brain's reaction to the stimulus event;
correlating the second series of images with the time-related information of the stimulus event; and
arranging and displaying the additional series of images in time sequence order next to the first series of images.

8. The method of claim 2, further comprising:
initiating the test session;
providing the plurality of stimulus events;
controlling the imaging apparatus; and
storing the time-related information of the plurality of stimulus events and the corresponding plurality of series of images in a non-volatile storage device for future use.

9. The method of claim 8, further comprising:
obtaining definitions of a maximum numerical value of a plurality of types of stimulus responses;
determining a point value and type of stimulus response for each of the actual responses of a test session;
calculating an evaluation subscore for each of the response types using the point values of the actual responses;
calculating an evaluation score for the session using the evaluation subscores;
repeating the lest session a plurality of times;
for each of the test sessions, calculating evaluation scores and a session score; and
comparing the session scores and the respective session subscores.

10. The method of claim 9, further comprising generating a report that includes at least a portion of the images, the session subscores, and the session scores.

11. A system for Event Related Imaging (ERI), comprising:
at least one brain imaging device;
a nonvolatile data storage device;
a processor operatively coupled to the brain imaging apparatus and the data storage device; and
a memory operatively coupled to the processor containing software executable by the processor, wherein by executing the software, the processor performs a method including automatedly:
recording time-related information of a stimulus event provided to a subject;
obtaining a series of images of the subject's brain's reaction to the stimulus event;
correlating the series of images with the time-related information of the stimulus event; and
arranging and displaying the series of images in time sequence order.

12. The system of claim 11, wherein the method executed by the processor running software further comprises automatedly:
obtaining a plurality of series of images of the subject's brain's reaction to a corresponding plurality of stimulus events of a test session;
generating an EEG graph during the test session; and
marking the time of each of the plurality of stimulus events on the EEG graph.

13. The system of claim 11, wherein the method executed by the processor running software further comprises automatedly:
generating the series of images by, at regular intervals beginning no later than the start of the stimulus event and continuing for a predetermined length of time:
generating an image and a corresponding time stamp; and
storing the image in conjunction with the time stamp; and
arranging the images by embedding each of the images in a spreadsheet in time sequence order in conjunction with the corresponding time stamp.

14. The system of claim 13, wherein the method executed by the processor running software further comprises automatedly:
generating each of the images from image data obtained at a corresponding one of the regular intervals; and
storing at least one of the image and the corresponding image data.

15. The system of claim 14, wherein the brain imaging device includes one or more electroencephalogram (EEG) sensors, infrared (IR) sensors, low resolution electromagnetic tomography (LORETA), standardized LORETA (sLORETA), computed tomography (CT) scanning, and functional Magnetic Resonance Imaging (fMRI), from which the data are obtained.

16. The system of claim 14, wherein the method executed by the processor running software further comprises automatedly:
receiving a selection of a region of interest of a brain image; and
for ones of the plurality of the brain images:
generating a copy of the brain image; and
cropping the copy in accordance with the region of interest; and
arranging the cropped images by embedding each of the cropped images in a spreadsheet in time sequence order in conjunction with the corresponding time stamp.

17. The system of claim 11, wherein the method executed by the processor running software further comprises automatedly:
obtaining a second series of images of the subject's brain's reaction to the stimulus event;
correlating the second series of images with the time-related information of the stimulus event; and
arranging and displaying the additional series of images in time sequence order next, to the first series of images.

18. The system of claim 12, wherein the method executed by the processor running software further comprises automatedly:
initiating the test session;
providing the plurality of stimulus events;
controlling the imaging apparatus; and
storing the time-related information of the plurality of stimulus events and the corresponding plurality of series of images in a non-volatile storage device for future use.

19. The system of claim 18, wherein the method executed by the processor running software further comprises automatedly:
obtaining definitions of a maximum numerical value of a plurality of types of stimulus responses;
determining a point value and type of stimulus response for each of the actual responses of a test session;
calculating an evaluation subscore for each of the response types using the point values of the actual responses;
calculating an evaluation score for the session using the evaluation subscores;
repeating the test session a plurality of times;
for each of the test sessions, calculating evaluation scores and a session score; and
comparing the session scores and the respective session subscores.

20. The system of claim 11, wherein the method executed by the processor running software further comprises automatedly:
generating a report that includes at least a portion of the images, the session subscores, and the session scores.
